(19) United States
(12) Patent Application Publication
(10) Pub. No.: US 2026/0350432 A1
(43) Pub. Date: Nov. 5, 2026

(54) CLINICAL EVENT DETECTION AND RECORDING SYSTEM USING ACTIVE LEARNING

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventor: Simon Mantha, New York, NY (US)

(73) Assignees: Memorial Sloan-Kettering Cancer Center, New York, NY (US); Memorial Hospital for Cancer and Allied Diseases, New York, NY (US); Sloan-Kettering Institute for Cancer Research, New York, NY (US)

(21) Appl. No.: 19/102,854

(22) PCT Filed: Aug. 9, 2023
(86) PCT No.: PCT/US2023/071956

§ 371 Date: Feb. 10, 2025

Related U.S. Application Data
(60) Provisional application No. 63/397,141, filed on Aug. 11, 2022.
(60) Provisional application No. 63/439,833, filed on Jan. 18, 2023.

(51) Int. Cl. G06N 5/04; G16H 50/20

(52) U.S. Cl. CPC G06N 5/04; G16H 50/20

(57) ABSTRACT

Clinical event detection and recording using active learning are disclosed. The claimed techniques include maintaining sequences of sentences. Each sequence of sequences is associated with an entity, and each sentence in each sequence is associated with a label and a timestamp. The techniques include iteratively training a machine-learning model using each sentence of the sequences as input and the label as a ground-truth value. Each iteration of training includes generating a prediction indicating whether the timestamp of each sentence occurred before or after an event of the entity. Each iteration of training includes validating the machine-learning model based on the prediction and the label of each sentence and the ground-truth value.

### CROSS-REFERENCES TO RELATED APPLICATIONS

This application claims the benefit of and priority to U.S. Provisional Patent Application No. 63/397,141, filed Aug. 11, 2022, and to U.S. Provisional Patent Application No. 63/439,833, filed Jan. 18, 2023, the contents of each of which are incorporated herein by reference in their entireties for all purposes.

### BACKGROUND

Clinical events, such as adverse clinical events, are important to document during the course of clinical trials and other tests. It is challenging to accurately and automatically record clinic events due to the lack of standardization in electronic medical records.

### SUMMARY

The systems and methods of the present disclosure provide techniques for clinical event detection and recording system using active (also referred to herein as adaptive) learning. The systems and methods described herein can utilize a subset of annotated electronic medical records to train one or more machine-learning models, which can then be executed over other, unlabeled sentences in electronic medical records to detect an occurrence of a clinical event, and to predict a time period during which the clinical event occurred. The techniques described herein can utilize an iterative and adaptive learning approach to train the machine-learning models. The techniques described herein drastically decrease the processing time of electronic medical records to evaluate the occurrence of adverse clinical events, and can allow fast, efficient and sensitive capture of clinical events, of a quality sufficient for reporting in scientific journals or for monitoring of clinical care for large hospital populations. The present techniques therefore provide a technical improvement to electronic medical records analysis systems by improving the computational efficiency and reducing the amount of workload required to generate annotated electronic medical records.

At least one aspect of the present disclosure relates to a method for clinical event detection and recording using adaptive learning. The method can be performed, for example, by one or more processors coupled to a non-transitory memory. The method can include maintaining a plurality of sequences of sentences. Each sequence of the plurality of sequences associated with an entity. Each sentence of the sequence associated with a label and a timestamp. The method can include iteratively training a machine-learning model using each sentence of the plurality of sequences as input and the label as a ground-truth value. Each iteration can include generating a prediction indicating whether the timestamp of each sentence occurred before or after a clinical event of the entity. Each iteration can include validating the machine-learning model based on the prediction and the label of each sentence of the plurality of sequences.

In some implementations, the label can indicate whether the timestamp of the sentence indicates it occurred before or after the clinical event of the entity. In some implementations, the method can include terminating training of the machine-learning model based on an output of validating the machine-learning model satisfying a threshold. In some implementations, the output can include one or more of a model specificity value, a model sensitivity value, a model precision value, or a model recall value. In some implementations, maintaining the plurality of sequences of sentences can include receiving, from a computing device, a first label for a first sentence of a sequence of sentences of the plurality of sequences of sentences.

In some implementations, iteratively training the machine-learning model can include receiving one or more labels for sentences of a second plurality of sentences. In some implementations, iteratively training the machine-learning model can include initiating a second iteration of training the machine-learning model using the second plurality of sentences and the one or more labels. In some implementations, the machine-learning model can include a natural language processing model.

In some implementations, the method can include executing the machine-learning model using a plurality of unlabeled sentences corresponding to the entity as input to generate a respective plurality of labels. In some implementations, the method can include determining a predicted timestamp of the clinical event involving the entity based on the respective plurality of labels. In some implementations, the method can include identifying, responsive to executing the machine-learning model, a second sentence corresponding to a second prediction that falls within an uncertainty threshold.

At least one other aspect of the present disclosure relates to a system configured for adaptive learning. The system can include one or more processors coupled to a non-transitory memory. The system can maintain a plurality of sequences of sentences. Each sequence of the plurality of sequences associated with an entity. Each sentence of the sequence associated with a label and a timestamp. The system can iteratively train a machine-learning model using each sentence of the plurality of sequences as input and the label as a ground-truth value. Each iteration can include generating a prediction indicating whether the timestamp of each sentence occurred before or after a clinical event of the entity. Each iteration can include validating the machine-learning model based on the prediction and the label of each sentence of the plurality of sequences.

In some implementations, the label can indicate whether the timestamp of the sentence indicates it occurred before or after the clinical event of the entity. In some implementations, the system can terminate training of the machine-learning model based on an output of validating the machine-learning model satisfying a threshold. In some implementations, the output can include one or more of a model specificity value, a model sensitivity value, a model precision value, or a model recall value. In some implementations, maintaining the plurality of sequences of sentences can include receiving, from a computing device, a first label for a first sentence of a sequence of sentences of the plurality of sequences of sentences.

In some implementations, iteratively training the machine-learning model can include receiving one or more labels for sentences of a second plurality of sentences. In some implementations, iteratively training the machine-learning model can include initiating a second iteration of training the machine-learning model using the second plurality of sentences and the one or more labels. In some implementations, the machine-learning model can include a natural language processing model.

In some implementations, the system can execute the machine-learning model using a plurality of unlabeled sentences corresponding to the entity as input to generate a respective plurality of labels. In some implementations, the system can determine a predicted timestamp of the clinical event involving the entity based on the respective plurality of labels. In some implementations, the system can identify, responsive to executing the machine-learning model, a second sentence corresponding to a second prediction that falls within an uncertainty threshold.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. It will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts an example system for clinical event detection and recording, in accordance with one or more implementations;

FIG. 2 depicts an example diagram showing multiple scenarios where a clinical event can occur during an clinical trial, in accordance with one or more implementations;

FIG. 3 depicts a flowchart for an example method of clinical event detection and recording, in accordance with one or more implementations; and

FIG. 4 is a block diagram of a server system and a client computer system in accordance with an illustrative embodiment'

FIG. 5 is a graph showing example experimental data relating to the detection of cancer-associated venous thromboembolism.

### DETAILED DESCRIPTION

The present techniques can allow for clinical event detection and recording. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. For the purpose of better understanding the present disclosure, a brief overview of the sections of the detailed description may be helpful:

Section A describes systems and methods for clinical event detection and recording; and

Section B describes a computing and network environment that may be utilized to implement the techniques described herein.

Section C describes the use of clinical event detection in the context of cancer-associated venous thromboembolism.

### A. Clinical Event Detection and Recording

The systems and methods described herein can be used to detect and predict the occurrence of clinical events in electronic medical records using adaptive learning. To do so, electronic medical records are stored in a database, which can be accessed to perform the techniques described herein. Each electronic medical record can include specific keywords potentially indicative of a clinical event (e.g. thrombotic episode, infection, bleeding, etc.). The sentences are generated through annotating of raw electronic medical record notes by a natural language processing model. The sentences can be arranged in chronological order, and upon detection of a pertinent clinical event in a sentence, the date and characteristics of the event are entered in the database. Sentences corresponding to an entity (e.g., a patient) appearing later than the event are not presented to the user, and the next record shown is that of the following patient.

Once a corpus of annotated list of notes or events have been gathered, one or more machine-learning models can be generated and trained to predict event dates for a similar, previously unlabeled corpus. Validity assessment can be performed by auditing a small sample of this second corpus. The machine-learning models can be iteratively trained using additionally provided annotated events and notes, and validation can be performed at each iteration until the model can predict the event dates for clinical events with sufficient accuracy (e.g., across multiple metrics such as specificity, recall, among others). The machine-learning models can then be executed over additional unlabeled electronic medical records to efficiently detect and predict timeframes for clinical events across various clinical trials.

The systems and methods of the present disclosure can be utilized in both research and quality assessment contexts. The present techniques can allow fast, efficient and sensitive capture of clinical events, of a quality sufficient for reporting in scientific journals or for monitoring of clinical care for large hospital populations. The present techniques therefore provide a technical improvement to electronic medical record analysis systems by improving the computational efficiency and reducing the amount of workload required to generate annotated electronic medical records. To provide such improvements and their corresponding benefits, the adaptive machine-learning models and methods used here (1) are based on computation of unique individual sentence probability labels trained on user annotations, (2) apply on-the-fly model training based on (a) model metrics with rational stopping point (for sensitivity etc.) and/or (b) efficient ambiguous sentence presentation (using Jaccard distance, etc.), and/or (3) use maximum likelihood estimation of event times based on individual sentence probability labels. These and other features are described in greater detail herein. Any suitable electronic medical record can be utilized with the techniques described herein, including, for example, clinical records and radiology reports.

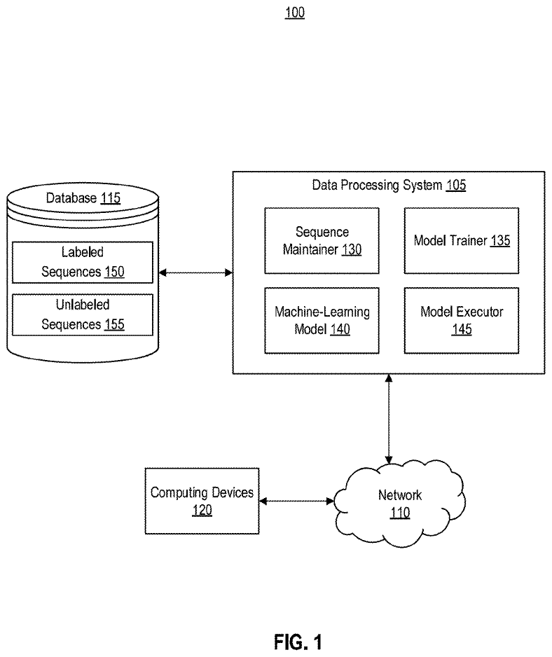

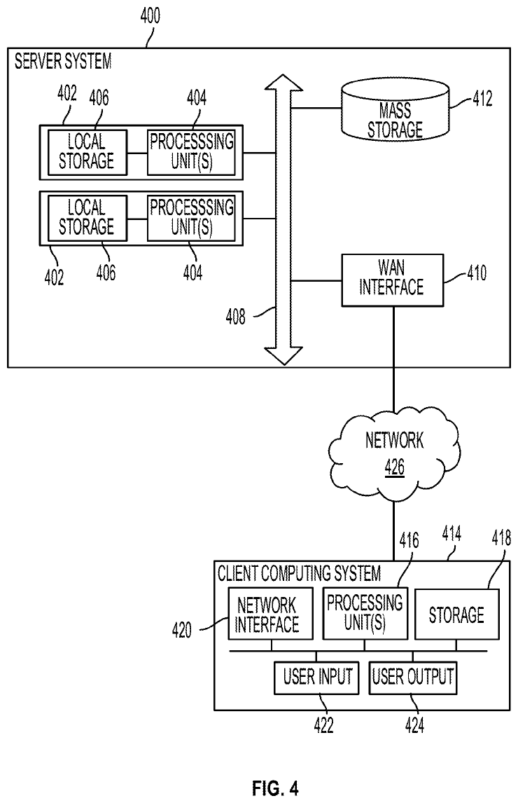

Referring now to FIG. 1, depicted is an example system for clinical event detection and recording using adaptive learning, in accordance with one or more implementations. In brief overview, the system 100 can include at least one data processing system 105, at least one network 110 (which may be the same as, or a part of, network 426 described herein below in conjunction with FIG. 4), a database 115, and a computing device 120. The data processing system 105 can include a sequence maintainer 130, a model trainer 135, a machine-learning model 140, and a model executor 145. The database can include one or more labeled sequences 130 and one or more unlabeled sequences 155. In some implementations, the data processing system 105 can include the database 115, and in some implementations, the database 115 can be external to the data processing system 105. For example, when the database 115 is external to the data processing system 105, the data processing system 105 (or the components thereof) can communicate with the database 115 via the network 110. In some implementations, the data processing system 105 can implement or perform any of the functionalities and operations discussed herein.

Each of the components (e.g., the data processing system 105, the network 110, the database 115, the computing device 120, the sequence maintainer 130, the model trainer 135, the machine-learning model 140, and the model executor 145, etc.) of the system 100 can be implemented using the hardware components or a combination of software with the hardware components of a computing system (e.g., server system 400, client computing system 414, any other computing system described herein, etc.) detailed herein in conjunction with FIG. 400. Each of the components of the data processing system 105 can perform the functionalities detailed herein.

In further detail, the data processing system 105 can include at least one processor and a memory (e.g., a processing circuit). The memory can store processor-executable instructions that, when executed by processor, cause the processor to perform one or more of the operations described herein. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 105 may include one or more computing devices or servers that can perform various functions as described herein. The data processing system 105 can include any or all of the components and perform any or all of the functions of the server system 400 or the client computing system 414 described herein below in conjunction with FIG. 4.

The network 110 can include computer networks such as the Internet, local, wide, metro or other area networks, intranets, satellite networks, other computer networks such as voice or data mobile phone communication networks, and combinations thereof. In some implementations, the network 110 can be, be a part of, or include one or more aspects of the network 426 described in connection with FIG. 4. The data processing system 105 of the system 100 can communicate via the network 110, for instance with at least one computing device 120. The network 110 can be any form of computer network that can relay information between the data processing system 105, the computing device 120, and in some implementations one or more external or third-party computing devices, such as web servers, among others. In some implementations, the network 110 can include the Internet and/or other types of data networks, such as a local area network (LAN), a wide area network (WAN), a cellular network, a satellite network, or other types of data networks.

The network 110 can also include any number of computing devices (e.g., computers, servers, routers, network switches, etc.) that are configured to receive and/or transmit data within the network 110. The network 110 can further include any number of hardwired and/or wireless connections. Any or all of the computing devices described herein (e.g., the data processing system 105, the computing device 120, the server system 400, the client computing device 414, etc.) can communicate wirelessly (e.g., via WiFi, cellular, radio, etc.) with a transceiver that is hardwired (e.g., via a fiber optic cable, a CAT5 cable, etc.) to other computing devices in the network 110. Any or all of the computing devices described herein (e.g., the data processing system 105, the computing device 120, the server system 400, the client computing device 414, etc.) can also communicate wirelessly with the computing devices of the network 110 via a proxy device (e.g., a router, network switch, or gateway).

The computing device 120 can include at least one processor and a memory, e.g., a processing circuit. The memory can store processor-executable instructions that, when executed by processor, cause the processor to perform one or more of the operations described herein. The processor may include a microprocessor, an ASIC, an FPGA, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions may include code from any suitable computer programming language. The computing device 120 can include one or more computing devices or servers that can perform various functions as described herein. The computing device 120 can include any or all of the components and perform any or all of the functions of the server system 400 or the client computing system 414 described herein below in conjunction with FIG. 4.

The computing device 120 can execute one or more applications, such as browser applications or web-based user interfaces, that facilitate communication with the data processing system 105 or the database 115 to perform the techniques described herein. For example, the computing device 120 can upload (e.g., transmit) one or more electronic medical records corresponding to one or more patients to the data processing system 105, which can store the records as part of the labeled sequences 150 or the unlabeled sequences 155 in the database 115. In some implementations, the computing device 120 can store the records as part of the labeled sequences 150 or the unlabeled sequences 155 in the database 115 via the network 110. As described herein, the computing device 120 can display one or more sentences, sequences, or corpora via a display device. Using the computer device 120, a user can utilize one or more graphical user interfaces provided by the data processing system 105 or the database 115 (e.g., in a webpage, web application, or application interface) to annotate the one or more sentences, sequences, or corpora with various labels described herein.

The database 115 can be a database configured to store and/or maintain any of the information described herein. The database 115 can maintain one or more data structures, which may contain, index, or otherwise store each of the values, pluralities, sets, variables, vectors, thresholds, or any generated or determined information described herein. The database 115 can be accessed using one or more memory addresses, index values, or identifiers of any item, structure, or region maintained in the database 115. The database 115 can be accessed by the components of the data processing system 105, or any other computing device described herein, via the network 110. In some implementations, the database 115 can be internal to the data processing system 105. In some implementations, the database 115 can be external to the data processing system 105, and may be accessed via the network 110. The database 115 can be distributed across many different computer systems or storage elements, and may be accessed via the network 110 or a suitable computer bus interface.

The data processing system 105 can store, in one or more regions of the memory of the data processing system 105, or in the database 115, the results of any or all computations, determinations, selections, identifications, generations, constructions, or calculations in one or more data structures indexed or identified with appropriate values. Any or all values stored in the database 115 may be accessed by any computing device described herein, such as the data processing system 105, to perform any of the functionalities or functions described herein.

The database 115 can store, in one or more data structures, one or more labeled sequences of sentences 150 (sometimes referred to herein as the “labeled sequences 150”). Each labeled sequence 150 can be associated with an identifier of an entity (e.g., a patient) to which the sequence of sentences corresponds. The labeled sequences 150 can include one or more sentences extracted from electronic medical records of the respective entity. Each labeled sequence 150 can further be stored in association with a corresponding clinical trial during which the electronic medical records were generated. The sentences in each labeled sequence 150 each be associated with a timestamp identifying the time that the sentence was recorded in the electronic medical record. Each labeled sequence 150 can be sorted based on the timestamp, such that the sentences in the labeled sequence 150 are arranged in chronological order (e.g., oldest to newest). As described herein, each of the sentences can include a predetermined keyword or phrase that could potentially relate to a medical issue, such as “thrombotic episode,” “infection,” “bleeding,” or the like.

In addition to a timestamp, each sentence in each labeled sequence can include a label. The label can indicate whether the sentence was recorded before or after an adverse clinical event involving the entity to which the label sequence 150 corresponds. The label may be a binary value (e.g., zero or one) indicating whether the sentence was recorded before or after an adverse clinical event involving the entity. An adverse clinical event can be any type of clinical event that may occur in a clinical trial, including any type of medical disease or condition. If a labeled sequence 150 is not associated with a clinical event, the label can indicate that all sentences occurred prior to a clinical event. If a labeled sequence 150 of an entity is associated with a clinical event, the labeled sequence 150 can be stored with an identifier of the clinical event.

The database 115 can store, in one or more data structures, one or more unlabeled sequences of sentences 154 (sometimes referred to herein as the “unlabeled sequences 155”). The unlabeled sequences 155 can be similar to the labeled sequences 150, except that the unlabeled sequences 155 are not labeled with a corresponding label that indicates whether each sentence was recorded before or after the occurrence of a clinical event. Like, the labeled sentences 150, each unlabeled sentence 155 can be associated with an identifier of an entity (e.g., a patient) to which the sequence of sentences corresponds. The unlabeled sequences 155 can include one or more sentences extracted from electronic medical records of the respective entity. Each unlabeled sequence 155 can further be stored in association with an identifier of a clinical trial during which the electronic medical records were generated.

The sentences in each unlabeled sequence 155 can each be associated with a timestamp identifying the time that the sentence was recorded in the electronic medical record. Each unlabeled sequence 155 can be sorted based on the timestamp, such that the sentences in the unlabeled sequence 155 are arranged in chronological order (e.g., oldest to newest). As described herein, each of the sentences can include a predetermined keyword or phrase that could potentially relate to a medical issue, such as “thrombotic episode,” “infection,” “bleeding,” or the like. Unlike the labeled sequences 150, each of the unlabeled sequences 155 are not stored in association with a timestamp identifying a time that a clinical event, if any, occurred. The data processing system 105 can execute the machine-learning model 140 to predict a timestamp corresponding to a clinical event, and store it in association with an unlabeled sequence 155, transforming it into a labeled sequence 150, as described herein.

Referring now to the operations of the data processing system 105, the sequence maintainer 130 can maintain one or more of sequences of sentences in the database 115, as the unlabeled sequences 155 and the labeled sequences 150. To store sentences, the sequence maintainer 130 can receive one or more electronic medical records from one or more external computing devices (e.g., the computing devices 120). The sequence maintainer 130 can scan through each of the electronic medical records for various entities, and can extract sentences that include at least one keyword or phrase of interest. The keywords or phrases of interest can be identified in a configurable lookup table, and can include, for example, words that may correspond to a clinical event, such as “thrombosis”, “clot”, “phlebitis.” Each extracted sentence can be stored in association with a respective timestamp identifying when the sentence was recorded in the electronic medical record (this value can also be extracted from the electronic medical record), an identifier of the entity to which the electronic medical record corresponds, and an identifier of the clinical trial to which the electronic medical record corresponds (if any). When extracted, the sentences can be sorted and assembled into a sequence of sentences for each entity, and stored as part of the unlabeled sequences 155.

To facilitate the adaptive learning techniques described herein to label the unlabeled sequences 155, the sequence maintainer 130 can receive annotations that act as ground-truth values for the machine-learning techniques described herein. To receive the annotations, the sequence maintainer 130 can communicate with the computing device 120 via the network 110 to receive annotations for one or more of the unlabeled sequences 155. The annotations can be transmitted in one or more messages from the computing device 120, and can include any type of label described herein. For example, the labels can include an indication of whether a sentence in an unlabeled sequence 155 was recorded in an electronic medical record prior to the occurrence of a clinical event. Likewise, an annotation may also include a timestamp indicating the time that a clinical event involving the corresponding entity occurred. The labels can be stored in association the corresponding unlabeled sequences 155, and the corresponding unlabeled sequences 150 can then be stored as part of the labeled sequences 150. The labeled sequences 150 can be used to train the machine-learning model 140.

In some implementations, the sequence maintainer 130 can generate one or more labels for the sentences in an unlabeled sequence 155 based on an annotation received from the computing device 120. For example, to establish an initial set of labeled sequences 150 from the unlabeled sequences 155, the sequence maintainer 130 can select an unlabeled sequence 155 from the database 115 (e.g., at random, based on a time period associated with unlabeled sequence 155, etc.), and transmit one or more of the sentences in the selected unlabeled sequence 155 to the computing device 120 (e.g., in predetermined order by timestamp). A user of the computing device 120 can either assign one or more of the labels associated with each sequence to the one or more sentences, or can provide a timestamp of the occurrence of a clinical event involving the entity associated with the unlabeled sequence 155. If the timestamp of the clinical event is provided, the sequence maintainer 130 can iterate through each of the sentences in the unlabeled sequence 155, which are ordered by timestamp, and assign a corresponding label indicating whether the respective sentence was recorded before or after the clinical event. The labels can be assigned to each sentence in the unlabeled sequence 155, which can then be stored as the labeled sequence 150. The sequence maintainer 130 can request additional annotations from the computing device 120 based on the iterative training of the machine-learning model 140.

The model trainer 135 can iteratively train the machine-learning model 140 using each sentence of the plurality of sequences as input and the label as a ground-truth value. The machine-learning model 140 can be any type of suitable machine-learning model, such as a classifier or regression model. Some non-limiting examples of the machine-learning model 140 can include a neural network (e.g., a convolutional neural network (CNN), a deep-neural network (DNN), etc.), a recurrent neural network (e.g., a long-short term memory (LSTM) model, etc.), regression classifiers (e.g., linear regression, sparse vector machine (SVM) models, etc.), or other types of classifiers (e.g., Naïve-Bayes classifiers, natural language processing (NLP) classifiers, etc.). In some implementations, the machine-learning model 140 can include more than one type of model, which may be executed sequentially or in parallel to generate any of the output data described herein.

Any suitable machine-learning algorithm or function can be utilized to train the machine learning models, including back-propagation for supervised learning algorithms. The machine-learning model 140 can be trained using the techniques described herein, including via adaptive learning, to generate an output that classifies whether a sentence in an unlabeled sequence 155 was recorded prior to or after the occurrence of a clinical event involving an entity associated with the unlabeled sequence 155. The machine-learning model 140 can generate an output confidence value, which indicates the confidence the generated output is accurate. The confidence value may be generated as a percentage value or a value from zero to one, with a value of ‘1.0’ indicating maximum confidence that the output is accurate, and a value of ‘0.0’ indicating minimum confidence that the output is accurate.

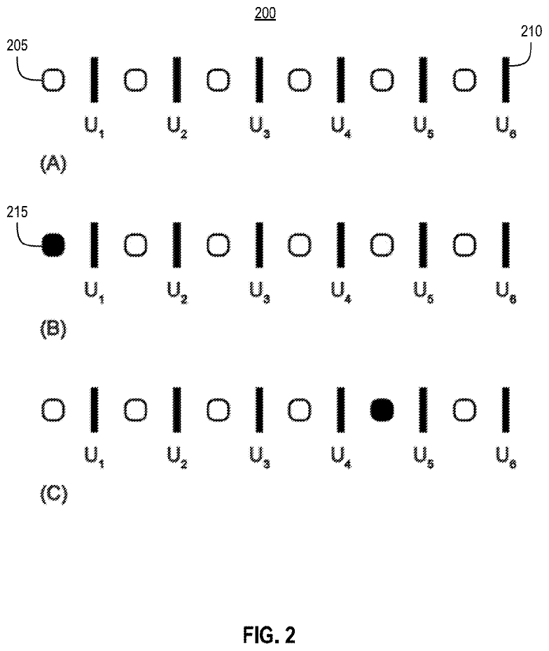

Prior to discussing the iterative machine-learning techniques of the present solution, describing the datasets utilized to train the model may be helpful. Referring to FIG. 2 in the context of the components of FIG. 1, illustrated is an example diagram 200 showing multiple scenarios (scenario A, B, and C) indicating different times that a clinical event can occur during an clinical trial, in accordance with one or more implementations. As described above, the database 115 stores labeled sequences 150 and unlabeled sequences 155. Each sentence in the labeled sequences 150 and the unlabeled sequences 155 can include at least one keyword of interest (e.g. “thrombosis”, “clot”, “phlebitis”), each sequence can be extracted from electronic medical records of a respective entity. The mathematical notation described herein is as follows. The training and test datasets include sequences (e.g., the labeled sequences 150 or the unlabeled sequences 155) of sentences (X₁, X₂, . . . Xₙ), where each sequence Xᵢ is derived from electronic medical record corpus i. Within a sequence Xᵢ, m sentences (Xᵢ₁, Xᵢ₂, . . . Xᵢₘ) have a corresponding timestamp Sᵢⱼ. Once a clinical event time Tᵢ has been recorded by a user of the computing device 120, the sequence maintainer 130 can labeled each sentence Xᵢⱼ for that entity as occurring before or after the event, where label z=0 if Sᵢⱼ<Tᵢ and z=1 if Sᵢⱼ>Tᵢ.

Using the techniques described herein, the machine-learning model 140 is trained to predict the z label for any given sentence. For example, “The patient was ruled out for deep vein thrombosis” can be classified as having a low probability of z=1. On the other hand, “She had a phlebitis 2 months ago” can score a high probability of z=1. For each sequence Xᵢ, there can be m timestamps Sᵢ, each corresponding to p date-time values U_{q}(shown in FIG. 2 as the time values 210) where p≤m (because two sentences may occur at the same time value 210 in the electronic medical records). Before each U_{q} value is a period 205 during which an event might have occurred, for a total ofp periods. Periods in which a clinical event occurred are shown in FIG. 2 as the clinical periods 215. There are thus p+1 possible scenarios, where scenario A shows that no event occurred during the study period, scenario B shows that an event occurred before the first timestamp (e.g., the time the first sentence was recorded in the electronic medical records), and scenario C shows that an event occurred between two timestamps.

After training, when the machine learning model 140 is applied to a given unlabeled electronic medical records of an entity, each sentence is assigned a probability of z=1 and a corresponding probability of z=0. For each event scenario, each sentence has probability P(Xᵢⱼ) of being consistent with the proposed event time. Based on these individual probabilities, a maximum likelihood estimation technique can be applied to find the event scenario which is most consistent with the observed model sentence-specific predictions (e.g., the scenario which maximizes IIP(Xᵢⱼ), etc.).

Referring back to FIG. 1, the machine model trainer 135 can iteratively train the machine-learning model 140 using the labeled sequences 150 as input. As described herein, the sequence maintainer 130 can transmit an initial set of unlabeled sequences 155 (or the sentences thereof) for annotation by a user of the computing device 120. The initial set of unlabeled sequences 155 can then be stored as an initial set of labeled sequences 150, which can be utilized in an iteration of the training techniques described herein. For each iteration of the training techniques, the sequence maintainer 130 can transmit additional sets of the unlabeled sequences 155 to the computing device 120 for annotation by the user, generating additional training data for the machine-learning model 140.

To train the machine-learning model 140, the model trainer 135 can execute a training and validation process, such as a k-fold cross-validation function. Cross-validation is a statistical method used to train and estimate the performance of machine learning models. The k-fold cross-validation process generally includes shuffling the labeled sequences 150 (but not the sequences therein) randomly, and then splitting the shuffled test set into k independent groups without replacement. Then, k−1 groups are used for model training (e.g., the training set), and one group is used for performance evaluation (e.g., the test set). This procedure is repeated k times (e.g., for k iterations) so that k performance estimates are obtained. The value k may be a predetermined or configurable value.

Each iteration can include executing the machine-learning model 140 to generate a prediction indicating whether the timestamp of each sentence occurred before or after a clinical event of the entity. If it is the first iteration of the training process, the model trainer 135 can generate one or more initial parameters for the model, for example, based on predetermined values relating to the data characteristics (e.g., size, format, etc.) of the labeled sequences 150. For example, the model trainer 135 can initiate the machine-learning model such that the machine learning model 135 can receive each of the sentences in each of the labeled sequences 150 as input, and generate a corresponding label that indicates whether the whether the timestamp of each sentence occurred before or after a clinical event of the entity. Initiating the model can include setting various parameters for the model based on predetermined and configurable hyperparameters, such as batch size, learning rate, or number of layers (if layers are utilized by the machine-learning model 140), or the like. The model trainer 135 can initialize one or more weight values or bias values to initial values prior to executing the first iteration of the training process.

At each iteration, the model trainer 135 can provide each of the sentences as input to the machine-learning model 135, and execute the model to propagate the input values and generate an output prediction value. The output prediction value can be trained to be equal to an estimate of whether the timestamp of each sentence occurred before or after a clinical event of the entity. The output value of the machine-learning model can be a scalar value, or a vector or another type of data structure with multiple output values. The output values for each sentence in each labeled sequence 150 in the training set can be stored as output values in the memory of the data processing system 105. The output values are then compared to the actual ground-truth value (e.g., the label) that is associated with each corresponding sentence in the labeled sequence 150. The comparison can be used to calculate a loss value, which is then propagated through the model to optimize the weights, biases, or other trainable parameters of the machine-learning model 140. To do so, the model trainer 135 can execute one or more supervised learning algorithms, such as a back-propagation algorithm implementing a type of gradient descent optimization function. The model trainer 135 can train the machine-learning model using each labeled sequence 150 in the training set for the current iteration.

After training the machine-learning model 140 using each labeled sequence 150 in the training set for the current iteration, the model trainer 135 can validate the machine-learning model 140 using each labeled sequence 150 in the test set for the current iteration. In doing so, the model trainer 135 can calculate various performance characteristics, including model sensitivity, specificity, precision, and recall, among others. The values can be calculated by comparing the predicted values of z to the actual values of z stored as labels in the respective labeled sequences 150. Precision is the ratio of true positives (e.g., the number of correctly predicted z=1 values) to total predicted positives (e.g., including the number of incorrectly predicted z=1 values, or false positives). To calculate the model precision, the model trainer 135 can calculate the total number of correctly predicted positive values (e.g., z=1) divided by the total number of correctly predicted positive values plus the number of incorrectly predicted positive values (e.g., incorrect predictions of z=1). Recall (or sensitivity) is the ratio of true positives to total (actual) positives in the data. To calculate the recall or sensitivity, the model trainer 135 can calculate the total number of correctly predicted positive values (e.g., z=1) divided by the total number of correctly predicted positive values plus the number of incorrectly predicted negative values (e.g., the number of incorrect predictions of z=0). Specificity is the ratio of true negatives to total negatives in the data. To calculate the specific of the machine-learning model 140, the model trainer 135 can calculate the total number of correctly predicted negative values (e.g., z=0) divided by the total number of correctly predicted negative values plus the number of incorrectly predicted positive values (e.g., the number of incorrect predictions of z=1).

After calculating the performance of the machine-learning model, the model trainer 135 can compare the performance values to predetermined thresholds for each of the performance metrics. Some example thresholds for the performance metrics can include 0.9 for model sensitivity, 0.95 for model precision, and 0.8 for model specificity. However, it should be understood that these are provided as examples, and that these values are configurable and can vary. The model trainer 135 can compare the calculated performance metrics to the thresholds. If the performance metrics are greater than the thresholds, the model training process can be terminated (e.g., as the performance can be a training termination condition).

Otherwise, the model trainer 135 can indicate that further training iterations are needed to train the model. As described herein, the model trainer 135 can request additional labeled sequences 150 from the computing device 120, by randomly selecting one or more of the unlabeled sequences 155 and transmitting the sentences of the unlabeled sequences 155 to computing device 120 for annotation by the user. Based on the annotation process described herein, the model trainer 135 can store additional labeled sequences 150 in the database 115. The additional labeled sequences 150 can be utilized in a subsequent iterations of the k-fold cross-validation fitting and validation processes described herein. The model trainer 135 can repeat the training and validation process, and request additional labeled sequences 150, until the performance metrics of the machine-learning model 140 satisfy the performance thresholds.

Once the machine-learning model 140 has been trained, the model executor 145 can execute the machine-learning model 140 using the unlabeled sequences 155 as input to generate respective labels (e.g., the respective prediction for the value z). As described above, the machine-learning model may output a confidence value (e.g., from zero to one) indicating the predicted accuracy of the output z value (e.g., using the notation above, the value P(Xᵢⱼ) of being accurate). In some implementations if a confidence value for an output value falls within an uncertainty value (or given range of uncertainty values, e.g., 0.40<P(Xij)<0.60, where P(Xij) corresponds to a prediction uncertainty value), the model executor 145 can transmit the corresponding input sentence to the computing device 120 for annotation by the user. Using the values generated by the machine-learning model 140 when providing each sentence of an unlabeled sequence 155 (corresponding to an entity) as input, the model executor 145 can determine a predicted timestamp of a clinical event involving the entity. To do so, the model executor 135 can execute the maximum likelihood estimation process. The maximum likelihood estimation process is a method of estimating the parameters of an assumed probability distribution, given some observed data (e.g., the estimated values of z for each sentence in the unlabeled sequence 155). Generally, the maximum likelihood estimation process maximizes a likelihood function such that, under an assumed statistical model, the predicted data is the most probable. The resulting likelihood function can then be used to estimate the timestamp of a clinical event involving the entity, if any.

Once the model executor 145 executes the machine-learning model and predicts an estimated timestamp for a clinical event indicated in each unlabeled sequence 155, the model executor 145 can present the estimated timestamp for the clinical event to the user in one or more graphical user interfaces. To do so, the model executor 145 can transmit display instructions (e.g., HTML5, JavaScript, other display instructions, etc.) to present the estimated event times for each unlabeled sequence 155 in a web-based or native-application-based graphical user interface at the computing device 120. The estimated event times may be presented, for example, with the sentences in the unlabeled sequence 155 that were recorded within a predetermined time range of the predicted timestamp of the clinical event. The estimated timestamp can be presented with an identifier of the entity associated with the clinical event, an identifier of the electronic medical record associated with the clinical event, or other relevant identifiers.

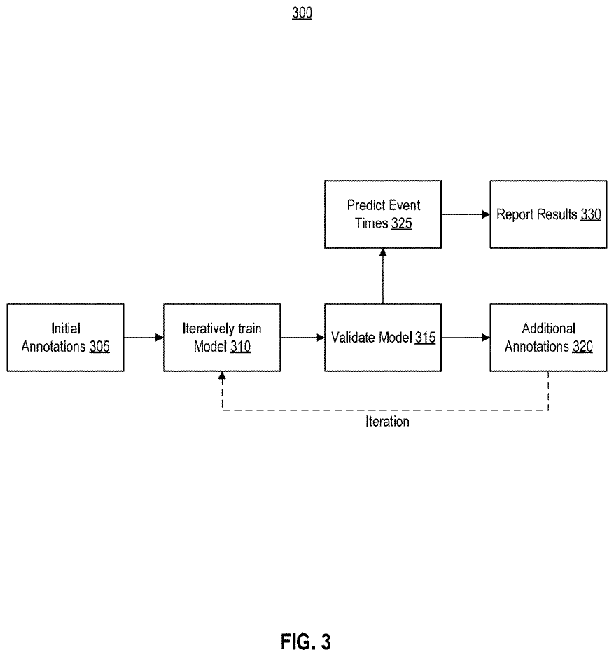

Referring now to FIG. 3, depicted is a flow diagram of an example method 300 of clinical event detection and recording, in accordance with one or more implementations. The method 300 can be performed, for example, by any computing device described herein, including the data processing system 105. In brief overview, in performing the method 300, the data processing system (e.g., the data processing system 105, etc.) can maintain initial annotations for sequences of sentences (STEP 305), iteratively train a machine-learning model (STEP 310), validate the machine-learning model (STEP 315), request additional annotations from a computing device (STEP 320), predict an estimated clinical event timestamp (STEP 325), and present the estimated clinical event timestamp (STEP 330).

In further detail, in performing the method 300, the data processing system (e.g., the data processing system 105, etc.) can maintain initial annotations for sequences of sentences (e.g., as the labeled sequences 150) (STEP 305). To do so, the sequence maintainer 130 can maintain one or more of sequences of sentences in a database (e.g., in the database 115 as the unlabeled sequences 155 and the labeled sequences 150). To store sentences, the data processing system can receive one or more electronic medical records from one or more external computing devices (e.g., the computing devices 120). The data processing system can scan through each of the electronic medical records for various entities, and can extract sentences that include at least one keyword or phrase of interest. The keywords or phrases of interest can be identified in a configurable lookup table, and can include, for example, words that may correspond to a clinical event, such as “thrombosis”, “clot”, “phlebitis.” Each extracted sentence can be stored in association with a respective timestamp identifying when the sentence was recorded in the electronic medical record (this value can also be extracted from the electronic medical record), an identifier of the entity to which the electronic medical record corresponds, and an identifier of the clinical trial to which the electronic medical record corresponds (if any). When extracted, the sentences can be sorted and assembled into a sequence of sentences for each entity, and stored as part of the unlabeled sequences.

To facilitate the adaptive learning techniques described herein to label the unlabeled sequences, the data processing system can receive annotations that act as ground-truth values for the machine-learning techniques described herein. To receive the annotations, the data processing system can communicate with a computing device (e.g., the computing device 120) via a network (e.g., the network 110) to receive annotations for one or more of the unlabeled sequences. The annotations can be transmitted in one or more messages from the computing device 120, and can include any type of label described herein. For example, the labels can include an indication of whether a sentence in an unlabeled sequence was recorded in an electronic medical record prior to the occurrence of a clinical event. Likewise, an annotation may also include a timestamp indicating the time that a clinical event involving the corresponding entity occurred. The labels can be stored in association the corresponding unlabeled sequences, and the corresponding unlabeled sequences can then be stored as part of the labeled sequences. The labeled sequences can be used to train a machine-learning model (e.g., the machine learning model 140).

In some implementations, the data processing system can generate one or more labels for the sentences in an unlabeled sequence based on an annotation received from the computing device 140. For example, to establish an initial set of labeled sequences from the unlabeled sequences, the data processing system can select an unlabeled sequence from the database (e.g., at random, based on a time period associated with unlabeled sequence, etc.), and transmit one or more of the sentences in the selected unlabeled sequence to the computing device 140 (e.g., in predetermined order by timestamp). A user of the computing device 140 can either assign one or more of the labels associated with each sequence to the one or more sentences, or can provide a timestamp of the occurrence of a clinical event involving the entity associated with the unlabeled sequence. If the timestamp of the clinical event is provided, the data processing system can iterate through each of the sentences in the unlabeled sequence, which are ordered by timestamp, and assign a corresponding label indicating whether the respective sentence was recorded before or after the clinical event. The labels can be assigned to each sentence in the unlabeled sequence, which can then be stored as the labeled sequence. The data processing system can request additional annotations from the computing device 140 based on the iterative training of the machine-learning model 140.

The data processing system can iteratively train a machine-learning model (STEP 310). The machine-learning model can be any type of suitable machine-learning model, such as a classifier or regression model. Some non-limiting examples of the machine-learning model can include a neural network (e.g., a CNN, a DNN, etc.), a recurrent neural network (e.g., an LSTM model, etc.), regression classifiers (e.g., linear regression, SVM models, etc.), or other types of classifiers (e.g., Naïve-Bayes classifiers, NLP classifiers, etc.). In some implementations, the machine-learning model can include more than one type of machine-learning model, which may be executed sequentially or in parallel to generate any of the output data described herein.

Any suitable machine-learning algorithm or function can be utilized to train the machine learning models, including back-propagation for supervised learning algorithms. The machine-learning model can be trained using the techniques described herein, including via adaptive learning, to generate an output that classifies whether a sentence in an unlabeled sequence was recorded prior to or after the occurrence of a clinical event involving an entity associated with the unlabeled sequence. The machine-learning model can generate an output confidence value, which indicates the confidence the generated output is accurate. The confidence value may be generated as a percentage value or a value from zero to one, with a value of ‘1.0’ indicating maximum confidence that the output is accurate, and a value of ‘0.0’ indicating minimum confidence that the output is accurate.

As described herein, the data processing system can transmit an initial set of unlabeled sequences (or the sentences thereof) for annotation by a user of the computing device. The initial set of unlabeled sequences can then be stored as an initial set of labeled sequences, which can be utilized in an iteration of the training techniques described herein. For each iteration of the training techniques, the data processing system can transmit additional sets of the unlabeled sequences to the computing device for annotation by the user, generating additional training data for the machine-learning model.

To train the machine-learning model, the data processing system can execute a training and validation process, such as a k-fold cross-validation function. Cross-validation is a statistical method used to train and estimate the performance of machine learning models. The k-fold cross-validation process generally includes shuffling the labeled sequences (but not the sequences therein) randomly, and then splitting the shuffled test set into k independent groups without replacement. Then, k−1 groups are used for model training (e.g., the training set), and one group is used for performance evaluation (e.g., the test set). This procedure is repeated k times (e.g., for k iterations) so that k performance estimates are obtained. The value k may be a predetermined or configurable value.

Each iteration can include executing the machine-learning model to generate a prediction indicating whether the timestamp of each sentence occurred before or after a clinical event of the entity. If it is the first iteration of the training process, the data processing system can generate one or more initial parameters for the model, for example, based on predetermined values relating to the data characteristics (e.g., size, format, etc.) of the labeled sequences. For example, the data processing system can initiate the machine-learning model such that the machine learning model can receive each of the sentences in each of the labeled sequences as input, and generate a corresponding label that indicates whether the whether the timestamp of each sentence occurred before or after a clinical event of the entity. Initiating the model can include setting various parameters for the model based on predetermined and configurable hyperparameters, such as batch size, learning rate, or number of layers (if layers are utilized by the machine-learning model), or the like. The data processing system can initialize one or more weight values or bias values to initial values prior to executing the first iteration of the training process.

At each iteration, the data processing system can provide each of the sentences as input to the machine-learning model, and execute the model to propagate the input values and generate an output prediction value. The output prediction value can be trained to be equal to an estimate of whether the timestamp of each sentence occurred before or after a clinical event of the entity. The output value of the machine-learning model can be a scalar value, or a vector or another type of data structure with multiple output values. The output values for each sentence in each labeled sequence in the training set can be stored as output values in the memory of the data processing system. The output values are then compared to the actual ground-truth value (e.g., the label) that is associated with each corresponding sentence in the labeled sequence. The comparison can be used to calculate a loss value, which is then propagated through the model to optimize the weights, biases, or other trainable parameters of the machine-learning model. To do so, the data processing system can execute one or more supervised learning algorithms, such as a back-propagation algorithm implementing a type of gradient descent optimization function. The data processing system can train the machine-learning model using each labeled sequence in the training set for the current iteration.

The data processing system can validate the machine-learning model (STEP 315). After training the machine-learning model using each labeled sequence in the training set for the current iteration, the data processing system can validate the machine-learning model using each labeled sequence in the test set for the current iteration. In doing so, the data processing system can calculate various performance characteristics, including model sensitivity, specificity, precision, and recall, among others. The values can be calculated by comparing the predicted values of z to the actual values of z stored as labels in the respective labeled sequences. Precision is the ratio of true positives (e.g., the number of correctly predicted z=1 values) to total predicted positives (e.g., including the number of incorrectly predicted z=1 values, or false positives). To calculate the model precision, the data processing system can calculate the total number of correctly predicted positive values (e.g., z=1) divided by the total number of correctly predicted positive values plus the number of incorrectly predicted positive values (e.g., incorrect predictions of z=1). Recall (or sensitivity) is the ratio of true positives to total (actual) positives in the data. To calculate the recall or sensitivity, the data processing system can calculate the total number of correctly predicted positive values (e.g., z=1) divided by the total number of correctly predicted positive values plus the number of incorrectly predicted negative values (e.g., the number of incorrect predictions of z=0). Specificity is the ratio of true negatives to total negatives in the data. To calculate the specific of the machine-learning model, the data processing system can calculate the total number of correctly predicted negative values (e.g., z=0) divided by the total number of correctly predicted negative values plus the number of incorrectly predicted positive values (e.g., the number of incorrect predictions of z=1).

After calculating the performance of the machine-learning model, the data processing system can compare the performance values to predetermined thresholds for each of the performance metrics. Some example thresholds for the performance metrics can include 0.9 for model sensitivity, 0.95 for model precision, and 0.8 for model specificity. However, it should be understood that these are provided as examples, and that these values are configurable and can vary. The data processing system can compare the calculated performance metrics to the thresholds. If the performance metrics are greater than the thresholds, the model training process can be terminated (e.g., as the performance can be a training termination condition), and the data processing system can execute STEP 325 of the method 300. Otherwise, the data processing system can indicate that further training iterations are needed to train the model, and can execute STEP 320.

The data processing system can request additional annotations from a computing device (STEP 320). As described herein, the data processing system can request additional labeled sequences from the computing device, by randomly selecting one or more of the unlabeled sequences and transmitting the sentences of the unlabeled sequences to computing device for annotation by the user. Based on the annotation process described herein, the data processing system can store additional labeled sequences in the database. The additional labeled sequences can be utilized in a subsequent iterations of the k-fold cross-validation fitting and validation processes described herein. The data processing system can repeat the training and validation process by then executing STEP 310 of the method 300.

The data processing system can predict an estimated clinical event timestamp (STEP 325). Once the machine-learning model has been trained, the data processing system can execute the machine-learning model using the unlabeled sequences as input to generate respective labels (e.g., the respective prediction for the value z). As described above, the machine-learning model may output a confidence value (e.g., from zero to one) indicating the predicted accuracy of the output z value (e.g., using the notation above, the value P(Xᵢⱼ) of being accurate). In some implementations if a confidence value for an output value falls within an uncertainty value (or range of values), the data processing system can transmit the corresponding input sentence to the computing device for annotation by the user. Using the values generated by the machine-learning model when providing each sentence of an unlabeled sequence (corresponding to an entity) as input, the data processing system can determine a predicted timestamp of a clinical event involving the entity. To do so, the data processing system can execute the maximum likelihood estimation process. The maximum likelihood estimation process is a method of estimating the parameters of an assumed probability distribution, given some observed data (e.g., the estimated values of z for each sentence in the unlabeled sequence). Generally, the maximum likelihood estimation process maximizes a likelihood function such that, under an assumed statistical model, the predicted data is the most probable. The resulting likelihood function can then be used to estimate the timestamp of a clinical event involving the entity, if any.

Where the confidence value falls within the uncertainty value (or range of values), the data processing system may transmit the corresponding input sentence to the computing device for annotation by the user in order to speed up model fitting by addressing the weakest (i.e., most uncertain) aspects of the model. In once such implementation, the model is used to run predictions on all unlabeled sentences (or sequences). The data processing system randomly presents sentences with the highest uncertainty value (e.g., closest to 0.5) to the user for annotation. In another implementation, unlabeled sentences (or sequences) may be selected using Jaccard distance or another suitable method rather than randomly in order to present more varied sentences to the user. In once such example, a first sentence (A) “She denied a history of DVT” and a second sentence (B) “He denies having had a deep vein thrombosis” are very similar, while a third sentence (C) “There was no PE noted on the CT” is quite different. In such an instance, in order to maximize learning speed for the model, the data processing system may present sentences (A) and (C) because of their differences but not sentences (A) and (B) given their similarity.

The data processing system can present the estimated timestamp of the clinical event (STEP 330). Once the data processing system executes the machine-learning model and predicts an estimated timestamp for a clinical event indicated in each unlabeled sequence, the data processing system can present the estimated timestamp for the clinical event to the user in one or more graphical user interfaces. To do so, the data processing system can transmit display instructions (e.g., HTML5, JavaScript, other display instructions, etc.) to present the estimated event times for each unlabeled sequence in a web-based or native-application-based graphical user interface at the computing device. The estimated event times may be presented, for example, with the sentences in the unlabeled sequence that were recorded within a predetermined time range of the predicted timestamp of the clinical event. The estimated timestamp can be presented with an identifier of the entity associated with the clinical event, an identifier of the electronic medical record associated with the clinical event, or other relevant identifiers.

### B. Computing and Network Environment

Various operations described herein can be implemented on computer systems. FIG. 4 shows a simplified block diagram of a representative server system 400, client computer system 414, and network 426 usable to implement certain embodiments of the present disclosure. In various embodiments, server system 400 or similar systems can implement services or servers described herein or portions thereof. Client computer system 414 or similar systems can implement clients described herein. The system 100 described herein can be similar to the server system 400. Server system 400 can have a modular design that incorporates a number of modules 402 (e.g., blades in a blade server embodiment); while two modules 402 are shown, any number can be provided. Each module 4s02 can include processing unit(s) 404 and local storage 406.

Processing unit(s) 404 can include a single processor, which can have one or more cores, or multiple processors. In some embodiments, processing unit(s) 404 can include a general-purpose primary processor as well as one or more special-purpose co-processors such as graphics processors, digital signal processors, or the like. In some embodiments, some or all processing units 404 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 404 can execute instructions stored in local storage 406. Any type of processors in any combination can be included in processing unit(s) 404.

Local storage 406 can include volatile storage media (e.g., DRAM, SRAM, SDRAM, or the like) and/or non-volatile storage media (e.g., magnetic or optical disk, flash memory, or the like). Storage media incorporated in local storage 406 can be fixed, removable or upgradeable as desired. Local storage 406 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 404 need at runtime. The ROM can store static data and instructions that are needed by processing unit(s) 404. The permanent storage device can be a non-volatile read-and-write memory device that can store instructions and data even when module 402 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 406 can store one or more software programs to be executed by processing unit(s) 404, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 404 cause server system 400 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 404. Software can be implemented as a single program or a collection of separate programs or program modules that interact as desired. From local storage 406 (or non-local storage described below), processing unit(s) 404 can retrieve program instructions to execute and data to process in order to execute various operations described above.

In some server systems 400, multiple modules 402 can be interconnected via a bus or other interconnect 408, forming a local area network that supports communication between modules 402 and other components of server system 400. Interconnect 408 can be implemented using various technologies including server racks, hubs, routers, etc.

A wide area network (WAN) interface 410 can provide data communication capability between the local area network (interconnect 408) and the network 426, such as the Internet. Technologies can be used, including wired (e.g., Ethernet, IEEE 802.3 standards) and/or wireless technologies (e.g., Wi-Fi, IEEE 802.11 standards).

In some embodiments, local storage 406 is intended to provide working memory for processing unit(s) 404, providing fast access to programs and/or data to be processed while reducing traffic on interconnect 408. Storage for larger quantities of data can be provided on the local area network by one or more mass storage subsystems 412 that can be connected to interconnect 408. Mass storage subsystem 412 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 412. In some embodiments, additional data storage resources may be accessible via WAN interface 410 (potentially with increased latency).

Server system 400 can operate in response to requests received via WAN interface 410. For example, one of modules 402 can implement a supervisory function and assign discrete tasks to other modules 402 in response to received requests. Work allocation techniques can be used. As requests are processed, results can be returned to the requester via WAN interface 410. Such operation can generally be automated. Further, in some embodiments, WAN interface 410 can connect multiple server systems 400 to each other, providing scalable systems capable of managing high volumes of activity. Other techniques for managing server systems and server farms (collections of server systems that cooperate) can be used, including dynamic resource allocation and reallocation.

Server system 400 can interact with various user-owned or user-operated devices via a wide-area network such as the Internet. An example of a user-operated device is shown in FIG. 4 as client computing system 414. Client computing system 414 can be implemented, for example, as a consumer device such as a smartphone, other mobile phone, tablet computer, wearable computing device (e.g., smart watch, eyeglasses), desktop computer, laptop computer, and so on.

For example, client computing system 414 can communicate via WAN interface 410. Client computing system 414 can include computer components such as processing unit(s) 416, storage device 418, network interface 420, user input device 422, and user output device 424. Client computing system 414 can be a computing device implemented in a variety of form factors, such as a desktop computer, laptop computer, tablet computer, smartphone, other mobile computing device, wearable computing device, or the like.

Processor 416 and storage device 418 can be similar to processing unit(s) 404 and local storage 406 described above. Suitable devices can be selected based on the demands to be placed on client computing system 414; for example, client computing system 414 can be implemented as a “thin” client with limited processing capability or as a high-powered computing device. Client computing system 414 can be provisioned with program code executable by processing unit(s) 416 to enable various interactions with server system 400.

Network interface 420 can provide a connection to the network 426, such as a wide area network (e.g., the Internet) to which WAN interface 410 of server system 400 is also connected. In various embodiments, network interface 420 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 422 can include any device (or devices) via which a user can provide signals to client computing system 414; client computing system 414 can interpret the signals as indicative of particular user requests or information. In various embodiments, user input device 422 can include any or all of a keyboard, touch pad, touch screen, mouse or other pointing device, scroll wheel, click wheel, dial, button, switch, keypad, microphone, and so on.

User output device 424 can include any device via which client computing system 414 can provide information to a user. For example, user output device 424 can include a display to display images generated by or delivered to client computing system 414. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 424 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 404 and 416 can provide various functionality for server system 400 and client computing system 414, including any of the functionality described herein as being performed by a server or client, or other functionality.

It will be appreciated that server system 400 and client computing system 414 are illustrative and that variations and modifications are possible. Computer systems used in connection with embodiments of the present disclosure can have other capabilities not specifically described here. Further, while server system 400 and client computing system 414 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### C. Natural Language Processing for the Detection of Cancer-Associated Venous Thromboembolism

The machine-learning approaches described herein can be utilized to label unlabeled medical records (e.g., unlabeled sequences 155), in order to identify the occurrence of medical events. Once such medical event that may be detected using the present techniques is venous thromboembolism (VTE), which may be determined based on electronic medical record corpora, radiology reports, and medical notes. When detecting VTE in medical notes, discrimination between events based on time of occurrence and association with cancer diagnosis can be performed.

As used herein, “venous thromboembolism” or “VTE” refers to a condition that occurs when a blood clot forms in a vein. VTE includes deep vein thrombosis (DVT) and pulmonary embolism (PE) episodes that occur at any time in the patient's history. Lower extremity DVT may include thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

The term “cancer-associated VTE” refers to pulmonary embolism or lower extremity deep vein thrombosis (DVT) that occurs in a subject after a cancer diagnosis or within the 365 days preceding a cancer diagnosis.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, or soft tissue sarcoma.

As used herein, the terms “subject”, “patient”, or “individual” can be an individual organism, a vertebrate, a mammal, or a human. In some embodiments, the subject, patient or individual is a human.

The term “Negative predictive value (NPV)” is defined as the proportion of subjects with a negative test result who are correctly identified. A high NPV means that when the test yields a negative result, it is unlikely that the result should have been positive. The NPV is determined as:

$$NPV = \frac{\# of True Negatives}{( \# of True Negatives + \# of False Positives )} = \frac{\# of True Negatives}{\# of Negative Calls} ,$$

where a “true negative” is the event that the test makes a negative prediction, and the subject has a negative result under the gold standard, and a “false negative” is the event that the test makes a negative prediction, and the subject has a positive result under the gold standard.

The term “positive predictive value (PPV),” or “precision rate” is a summary statistic used to describe the proportion of subjects with positive results who are correctly identified. It is a measure of the performance of a predictive method, as it reflects the probability that a positive result reflects the underlying condition being tested for. Its value does however depend on the prevalence of the outcome of interest, which may be unknown for a particular target population. The PPV can be derived using Bayes' theorem. The PPV is defined as:

$$PPV = \frac{\# of True Positives}{( \# of True Positives + \# of False Positives )} = \frac{\# of True Positives}{\# of Positives Calls} ,$$

where a “true positive” is the event that the predictive test makes a positive prediction, and the subject has a positive result under the gold standard, and a “false positive” is the event that the test makes a positive prediction, and the subject has a negative result under the gold standard.

If the prevalence, sensitivity, and specificity are known, the positive and negative predictive values (PPV and NPV) can be calculated for any prevalence as follows:

$$PPV = \frac{sensitivity \times prevalence}{sensitivity \times prevalence + ( 1 - specificity ) \times ( 1 - prevalence )}$$

$$NPV = \frac{specificity \times ( 1 - prevalence )}{( 1 - sensitivity ) \times prevalence + specificity \times ( 1 - prevalence )}$$

If the prevalence of the disease is very low, the positive predictive value will not be close to 1, even if both the sensitivity and specificity are high. Thus in screening the general population it is inevitable that many people with positive test results will be false positives. The rarer the abnormality, the higher the certainty that a negative test indicates no abnormality, and the lower the certainty that a positive result truly indicates an abnormality. The prevalence can be interpreted as the probability before the test is carried out that the subject has the disease, known as the prior probability of disease. The positive and negative predictive values are the revised estimates of the same probability for those subjects who are positive and negative on the test, and are known as posterior probabilities. The difference between the prior and posterior probabilities is one way of assessing the usefulness of the test.

For any test result, one can compare the probability of obtaining that result if the patient truly had the condition of interest with the corresponding probability if he or she were healthy. The ratio of these probabilities is called the likelihood ratio, calculated as sensitivity/(1−specificity).

In statistics, sensitivity and specificity are statistical measures of the performance of a binary classification test. The term “sensitivity” (also called “recall rate”) measures the proportion of actual positives which are correctly identified as such (e.g. the percentage of subjects who are correctly identified as having a condition). Sensitivity relates to the ability of a predictive test to identify positive results and is computed as the number of true positives divided by the sum of the number of true positives and the number of false negatives. The term “specificity” measures the proportion of negatives which are correctly identified (e.g., the percentage of subjects who are correctly identified as not having the condition). Specificity relates to the ability of a predictive test to identify negative results and is computed as the number of true negatives divided by the sum of the number of true negatives and the number of false positives. Sensitivity and specificity are closely related to the concepts of type I and type II errors. A theoretical, optimal prediction aims to achieve 100% sensitivity and 100% specificity, however theoretically any predictor will possess a minimum error bound known as the Bayes error rate.

For any test, there is usually a trade-off between sensitivity and specificity, which can be represented graphically using a receiver operating characteristic (ROC) curve. In some embodiments, a ROC is used to generate a summary statistic. Some common versions are: the intercept of the ROC curve with the line at 90 degrees to the no-discrimination line (also called Youden's J statistic); the area between the ROC curve and the no-discrimination line; the area under the ROC curve, or “AUC” (“Area Under Curve”), or A′ (pronounced “a-prime”); d′ (pronounced “d-prime”), the distance between the mean of the distribution of activity in the system under noise-alone conditions and its distribution under signal-alone conditions, divided by their standard deviation, under the assumption that both these distributions are normal with the same standard deviation. Under these assumptions, it can be proved that the shape of the ROC depends only on d′.

In an example experiment, training, validation, development, and testing sets were derived in a 7:1:1:1 ratio from a cohort of 35,391 patients. Both clinical notes and radiology reports were labelled as occurring before or after a cancer-associated VTE event. Notes and reports including keywords drawn from a specific clinically meaningful set pertaining to VTE were included. VTE was defined as the presence of lower extremity deep vein thrombosis or pulmonary embolism. Thrombotic episodes were considered associated with cancer if they occurred no earlier than one year before cancer diagnosis. Neural network training (e.g., the training of the machine-learning model 140), in this non-limiting example experiment, was performed using PyTorch 1.13.1 using the “longformer-large-4096” model (e.g., a transformer model) in a cloud computing environment. Training the model was performed using techniques similar to those described herein, for example, in connection with the model trainer 135. A global attention mask was established based on tokens derived from the clinically meaningful lexicon. Two tasks were evaluated, including the detection of VTE events or the detection of only cancer-associated VTE episodes. The development set was used for final assessment of model metrics.

As described herein, the machine learning model outputs the probability of any given note as coming after a clinical event. In this example, after scoring by the model, each patient has a set of n probability values {p₁, p₂, . . . pₙ}, each being the output of a clinical note/report (e.g., a sequence) evaluated by the machine-learning model (e.g., an NLP model, etc.). Post-processing is performed on the sets of probability values to determine whether a particular event occurred at the patient level. In this specific example, the highest p for each patient was chosen, and applied to a threshold to determine whether the patient was labeled as “VTE” or “no VTE.”

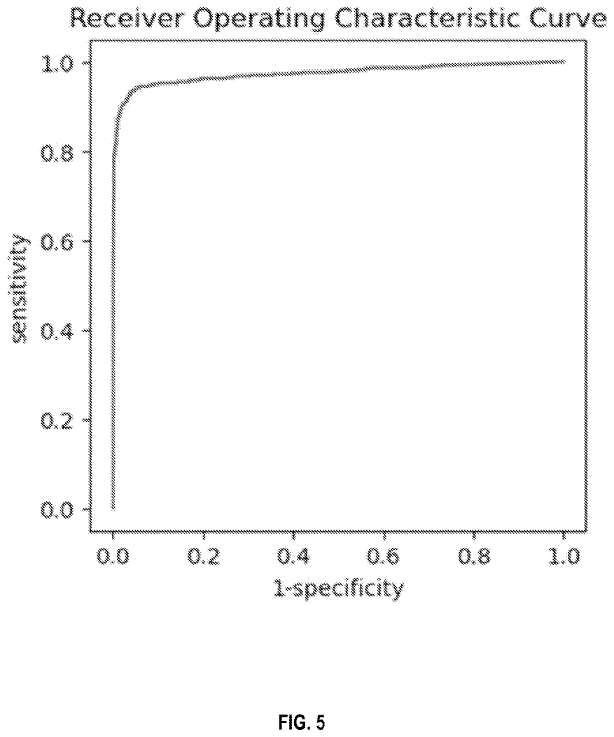

In this non-limiting example experiment, 394,948 documents retrieved from the electronic medical records (EMR) of 24,774 patients were included in the set of training data used to train the machine-learning model. Discriminatory power for detection of cancer-associated VTE at the patient level was excellent for cancer-associated VTE in the validation set (AUROC=0.974, 95% CI=0.964-0.984; see FIG. 5). Using a detection threshold based on the validation set, sensitivity and specificity were 0.935 (0.916-0.954) and 0.974 (0.967-0.981) for the model tasked with detecting any VTE event, compared to 0.908 (0.873-0.943) and 0.976 (0.971-0.982) respectively for the model trained to detect only cancer-associated VTE, as shown in the Table below.

| | Accuracy | Precision | Recall | Specificity |
|---|---|---|---|---|
| Any VTE* | 0.966 | 0.87 | 0.935 | 0.974 |
| | (0.959-0.973) | (0.844-0.895) | (0.916-0.954) | (0.967-0.981) |
| Cancer- | 0.966 | 0.862 | 0.908 | 0.976 |
| associated | (0.959-0.974) | (0.828-0.895) | (0.873-0.943) | (0.971-0.982) |
| VTE only† | | | | |

The results in the Table above are provided as mean metric value with 95% confidence interval, estimated with bootstrapping. *Any lower extremity deep vein thrombosis or pulmonary embolism episode at any time in the patient's history. †Lower extremity deep vein thrombosis or pulmonary embolism occurring from one year before cancer diagnosis to any time later.

FIG. 5 shows an example plot of the sensitivity and specificity for a full range of thresholds. The plot in FIG. 5 shows an ROC for this example experiment. The machine-learning techniques described herein perform competitively when utilizing clinical notes, radiology reports, and other clinical records or EMR. Other types of post-processing can be utilized, such as a maximum likelihood algorithm. In this example experiment, a transformer-based NLP model was derived and applied successfully to accurately identify patients with cancer-associated VTE. The loss of performance compared to detection of VTE regardless of association with cancer was not significant.

The various neural networks and/or machine-learning models described herein may include any type of large language model(s). Although some implementations may utilize a longformer-4096 model (e.g., “longformer-large-4096” model, a transformer model, etc.), it should be understood that any type of generative model may also be used. In some implementations, the systems and methods described herein may be provide options for prompt engineering to identify the best text input for the model to extract event-specific information from electronic health record documents. Reinforcement learning based on misclassified cases may also be used at to improve model performance.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

Aspects can be combined and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using a suitable apparatus, which can take the form of one or more programmable computers running computer programs arranged to implement the aspect. As used in the specification and in the claims, the singular form of ‘a’, ‘an’, and ‘the’ include plural referents unless the context clearly dictates otherwise.

What is claimed is:

1. A method, comprising:
maintaining, by one or more processors coupled to a non-transitory memory, a plurality of sequences of sentences, each sequence of the plurality of sequences associated with an entity, each sentence of the sequence associated with a label and a timestamp;
iteratively training, by the one or more processors, a machine-learning model using each sentence of the plurality of sequences as input and the label as a ground-truth value, wherein each iteration comprises:
generating a prediction indicating whether the timestamp of each sentence occurred before or after a clinical event of the entity; and
validating the machine-learning model based on the prediction and the label of each sentence of the plurality of sequences.

2. The method of claim 1, wherein the label indicates whether the timestamp of the sentence indicates occurred before or after the clinical event of the entity.

3. The method of claim 1, further comprising terminating, by the one or more processors, training of the machine-learning model based on an output of validating the machine-learning model satisfying a threshold.

4. The method of claim 3, wherein the output comprises one or more of a model specificity value, a model sensitivity value, a model precision value, or a model recall value.

5. The method of claim 1, wherein maintaining the plurality of sequences of sentences comprises receiving, by the one or more processors from a computing device, a first label for a first sentence of a sequence of sentences of the plurality of sequences of sentences.

6. The method of claim 1, wherein iteratively training the machine-learning model comprises:
receiving, by the one or more processors, one or more labels for sentences of a second plurality of sentences; and
initiating, by the one or more processors, a second iteration of training the machine-learning model using the second the second plurality of sentences and the one or more labels.

7. The method of claim 1, wherein the machine-learning model comprises a natural language processing model.

8. The method of claim 1, further comprising executing, by the one or more processors, the machine-learning model using a plurality of unlabeled sentences corresponding to the entity as input to generate a respective plurality of labels.

9. The method of claim 1, further comprising determining, by the one or more processors, a predicted timestamp of the clinical event involving the entity based on the respective plurality of labels.

10. The method of claim 1, further comprising:
identifying, by the one or more processors, responsive to executing the machine-learning model, a second sentence corresponding to a second prediction that falls within an uncertainty threshold;
responsive to the second prediction falling within the uncertainty threshold, presenting the second sentence to a user for annotation;
receiving the annotation for the second sentence from the user; and
updating the machine-learning model based on the received annotation for the second sentence.

11. A system, comprising:
one or more processors coupled to a non-transitory memory, the one or more processors configured to:
maintain a plurality of sequences of sentences, each sequence of the plurality of sequences associated with an entity, each sentence of the sequence associated with a label and a timestamp;
iteratively train a machine-learning model using each sentence of the plurality of sequences as input and the label as a ground-truth value, wherein each iteration comprises:
generating a prediction indicating whether the timestamp of each sentence occurred before or after a clinical event of the entity; and
validating the machine-learning model based on the prediction and the label of each sentence of the plurality of sequences.

12. The system of claim 11, wherein the label indicates whether the timestamp of the sentence indicates occurred before or after the clinical of the entity.

13. The system of claim 11, wherein the one or more processors are further configured to terminate training of the machine-learning model based on an output of validating the machine-learning model satisfying a threshold.

14. The system of claim 13, wherein the output comprises one or more of a model specificity value, a model sensitivity value, a model precision value, or a model recall value.

15. The system of claim 11, wherein the one or more processors are further configured to receive, from a computing device, a first label for a first sentence of a sequence of sentences of the plurality of sequences of sentences.

16. The system of claim 11, wherein the one or more processors are further configured to iteratively train the machine-learning model by performing operations comprising:
receiving one or more labels for sentences of a second plurality of sentences; and
initiating a second iteration of training the machine-learning model using the second the second plurality of sentences and the one or more labels.

17. The system of claim 11, wherein the machine-learning model comprises a natural language processing model.

18. The system of claim 11, further comprising executing the machine-learning model using a plurality of unlabeled sentences corresponding to the entity as input to generate a respective plurality of labels.

19. The system of claim 11, further comprising determining a predicted timestamp of the clinical event involving the entity based on the respective plurality of labels.

20. The system of claim 11, further comprising identifying, responsive to executing the machine-learning model, a second sentence corresponding to a second prediction that falls within an uncertainty threshold.

21. A method, comprising:
executing, by one or more processors coupled to memory, a machine-learning model using a plurality of sentences derived from at least one electronic medical record of a patient as input, the machine-learning model generating a set of probability values indicating whether each sentence of the plurality of sentences followed a cancer-associated venous thromboembolism (VTE) event;
generating, by the one or more processors, a score for the patient based on the set of probability values; and
determining, by the one or more processors, whether the patient experienced a cancer-associated VTE event based on the score.
